(19) United States
(12) Patent Application Publication
(10) Pub. No.: US 2003/0157642 A1
(43) Pub. Date: Aug. 21, 2003

(54) Increasing production of proteins in gram-positive microorganisms

(76) Inventors: Robert M. Caldwell, Belmont, CA (US); Wilhelmus J. Quax, Kropswolde (NL)

Correspondence Address: KAMRIN T. MACKNIGHT, GENENCOR INTERNATIONAL, INC., 925 PAGE MILL ROAD, PALO ALTO, CA, 94034-1013, US

(21) Appl. No.: 10/310,507

(22) Filed: Dec. 4, 2002

Related U.S. Application Data
(63) Continuation-in-part of application No. 09/462,843, filed on Mar. 22, 2000, now Pat. No. 6,506,579.

(30) Foreign Application Priority Data
Jul. 15, 1997 (EP) EP 97305288.5
Jul. 14, 1998 (WO) PCT/US98/14648

(51) Int. Cl. C12P 21/02; C12N 1/21; C07H 21/04; C12N 15/74

(52) U.S. Cl. 435/69.1; 435/252.31; 435/320.1; 536/23.2

(57) ABSTRACT

The present invention relates to secretion in Gram-positive microorganisms. The present invention provides the nuclei acid and amino acid sequences for the Bacillus subtilis secretion factor SecG. The present invention also provides means for increasing the secretion of heterologous or homologous proteins in Gram-positive microorganisms.

The present application is a Continuation-in-Part of and claims priority to U.S. patent application Ser. No. 09/462,843, filed Mar. 22, 2000, which claims priority to PCT/US98/14648, filed Jul. 14, 1998 and EP 97305288.5, filed Jul. 15, 1997.

### FIELD OF THE INVENTION

The present invention generally relates to expression of proteins in Gram-positive microorganisms and specifically to the Gram-positive microorganism secretion factor, SecG. The present invention also provides expression vectors, methods and systems for the production of proteins in Gram-positive microorganisms.

### BACKGROUND OF THE INVENTION

Gram-positive microorganisms, such as members of the genus Bacillus, have been used for large-scale industrial fermentation due, in part, to their ability to secrete their fermentation products into the culture media. In Gram-positive bacteria, secreted proteins are exported across a cell membrane and a cell wall, and then are subsequently released into the external media usually obtaining their native conformation. Previously identified secretion factors from Gram-positive microorganisms include SecA (Sadaie et al., Gene 98:101-105 [1991]), SecY (Suh et al., Mol. Microbiol., 4:305-314 [1990]), SecE (Jeong et al., Mol. Microbiol., 10:133-142 [1993]), FtsY and FfH (PCT/NL 96/00278), as well as PrsA (WO 94/19471).

In contrast, in the Gram-negative microorganism, E. coli, protein is transported to the periplasm rather than across the cell membrane and cell wall and into the culture media. E. coli has at least two types of components of the secretory mechanism, soluble cytoplasmic proteins and membrane associated proteins. Reported E. coli secretion factors include the soluble cytoplasmic proteins, SecB and heat shock proteins; the peripheral membrane-associated protein SecA; and the integral membrane proteins SecY, SecE, SecD and SecF.

In spite of advances in understanding portions of the protein secretion machinery in prokaryotic cells, the complete mechanism of protein secretion, especially in Gram-positive microorganisms, such as Bacillus, has yet to be fully elucidated.

### SUMMARY OF THE INVENTION

The present invention generally relates to expression of proteins in Gram-positive microorganisms and specifically to the Gram-positive microorganism secretion factor, SecG. The present invention also provides expression vectors, methods and systems for the production of proteins in Gram-positive microorganisms.

In some embodiments, the present invention provides expression vectors comprising a nucleic acid sequence encoding a secretion factor G (SecG) protein, wherein the secretion factor G is under the control of an expression signal capable of overexpressing the secretion factor in a Gram-positive microorganism, and wherein the nucleic acid sequence comprises SEQ ID NO: 1. In some preferred embodiments, the Gram-positive microorganism is a member of the genus Bacillus. In some particularly preferred embodiments, the member of the genus Bacillus is selected from the group consisting of B. subtilis, B. licheniformis, B. lentus, B. brevis, B. stearothermophilus, B. alcalophilus, B. amyloliquefaciens, B. coagulans, B. circulars, B. lautus, and B. thuringiensis. In further embodiments, the present invention provides Gram-positive microorganisms (i.e., host cells) comprising the expression vector. In some preferred embodiments, the Gram-positive microorganism is a member of the genus Bacillus. In some particularly preferred embodiments, the host cell is a member of the genus Bacillus is selected from the group consisting of B. subtilis, B. licheniformis, B. lentus, B. brevis, B. stearothermophilus, B. alcalophilus, B. arnyloliquefaciens, B. coagulans, B. circulars, B. lautus, and B. thuringiensis. In some embodiments, the host cell further expresses at least one heterologous protein. In some preferred embodiments, the heterologous protein is selected from the group consisting of hormones, enzymes, growth factors, and cytokines. In some particularly preferred embodiments, the heterologous protein is an enzyme. In further embodiments, the enzyme is selected from the group consisting of proteases, cellulases, amylases, carbohydrases, lipases, reductases, isomerases, epimerases, tautomerases, transferases, kinases, and phosphatases.

The present invention also provides methods for secreting proteins from Gram-positive microorganisms, comprising the steps of obtaining a Gram-positive microorganism host cell comprising nucleic acid sequence encoding a secretion factor G (SecG) protein, wherein the nucleic acid sequence comprises the nucleic acid sequence set forth in SEQ ID NO: 1 and the nucleic acid sequence is under the control of an expression signal capable of expressing SecG in a Gram-positive microorganism and further comprising a nucleic acid sequence encoding the protein to be secreted; and culturing the microorganism under conditions suitable for expression of SecG and expression and secretion of the protein. In some embodiments, the Gram-positive microorganism also comprises nucleic acid encoding at least one additional secretion factor selected from the group consisting of secretion factor Y (SecY), secretion factor E (SecE) and secretion factor A (SecA). In further embodiments, the protein is homologous to the host cell. In some preferred embodiments, the Gram-positive microorganism is a member of the genus Bacillus. In some preferred embodiments, the member of the genus Bacillus is selected from the group consisting of B. subtilis, B. licheniformis, B. lentus, B. brevis, B. stearothermophilus, B. alcalophilus, B. amyloliquefaciens, B. coagulans, B. circulans, B. lautus, and Bacillus thuringiensis. In alternative preferred embodiments, the Bacillus expresses at least one heterologous protein selected from the group consisting of hormones, enzymes, growth factors, and cytokines. In some particularly preferred embodiments, the heterologous protein is an enzyme. In further embodiments, the enzyme is selected from the group consisting of proteases, cellulases, amylases, carbohydrases, lipases, reductases, isomerases, epimerases, tautomerases, transferases, kinases, and phosphatases.

The present invention further provides expression vectors comprising a nucleic acid sequence encoding a secretion factor G (SecG) protein comprising the amino acid sequence set forth in SEQ ID NO: 2, wherein the secretion factor G is under the control of expression signals capable of overexpressing the secretion factor in a Gram-positive microorganism, and wherein the nucleic acid sequence comprises SEQ ID NO: 1.

The present invention also provides methods for secreting a protein in a Gram-positive microorganism comprising the steps of obtaining a Gram-positive microorganism host cell comprising nucleic acid sequence encoding a secretion factor G (SecG) protein, wherein the nucleic acid sequence comprises the nucleic acid sequence set forth in SEQ ID NO: 1 and the nucleic acid sequence is under the control of expression signals capable of expressing SecG in a Gram-positive microorganism and further comprising nucleic acid encoding the protein; and culturing the microorganism under conditions suitable for expression of SecG and expression and secretion of the protein, wherein the protein comprises the amino acid sequence set forth in SEQ ID NO: 2.

The present invention further provides Gram-positive microorganisms encoding a mutated Shine Delgarno sequence such that the translation of the transcript comprising secretion factor G (SecG) is modulated. In some preferred embodiments, the Gram-positive microorganism is a member of the genus Bacillus. In some particularly preferred embodiments, member of the genus Bacillus is selected from the group consisting of B. subtilis, B. licheniformis, B. lentus, B. brevis, B. stearothermophilus, B. alkalophilus, B. amyloliquefaciens, B. coagulans, B. circulans, B. lautus and B. thuringiensis. In some embodiments, the modulation comprises increasing the expression of SecG, while in alternative embodiments the modulation comprises decreasing the expression of SecG. In still further embodiments, the microorganism is capable of expressing at least one heterologous protein. In some embodiments, the heterologous protein is selected from the group consisting of hormones, enzymes, growth factors, and cytokines. In some particularly preferred embodiments, the heterologous protein is an enzyme. In some embodiments, the enzyme is selected from the group consisting of a proteases, cellulases, amylases, carbohydrases, lipases, reductases, isomerases, epimerases, tautomerases, transferases, kinases, and phosphatases.

The present invention also provides Gram-positive microorganisms encoding a mutated RNA polymerase sigma factor alpha (σ_{A}) sequence such that the expression of secretion factor G (SecG) is modulated. In some preferred embodiments, the Gram-positive microorganism is a member of the genus Bacillus. In some particularly preferred embodiments, the Bacillus is selected from the group consisting of B. subtilis, B. licheniformis, B. lentus, B. brevis, B. stearothermophilus, B. alkalophilus, B. amyloliquefaciens, B. coagulans, B. circulans, B. lautus and B. thuringiensis. In some embodiments, the modulation comprises increasing the expression of SecG, while in other embodiments, the modulation comprises decreasing the expression of SecG. In still further embodiments, the Gram-positive microorganisms are capable of expressing at least one heterologous protein. In some preferred embodiments, the heterologous protein is selected from the group consisting of hormone, enzyme, growth factor and cytokines. In some particularly preferred embodiments, the heterologous protein is an enzyme. In alternative preferred embodiments, the enzyme is selected from the group consisting of a proteases, cellulases, amylases, carbohydrases, lipases, reductases, isomerases, epimerases, tautomerases, transferases, kinases, and phosphatases.

The present invention further provides methods for secreting a protein in a Gram-positive microorganism comprising the steps of obtaining a Gram-positive microorganism host cell comprising nucleic acid encoding SecG wherein the nucleic acid is under the control of expression signals capable of expressing SecG in a Gram-positive microorganism and further comprising nucleic acid encoding the protein; and culturing the microorganism under conditions suitable for expression of SecG and expression and secretion of the protein. In some embodiments, the microorganism further comprises nucleic acid encoding at least one additional secretion factor selected from the group consisting of SecY, SecE and SecA. In some preferred embodiments, the protein is homologous to the host cell, while in other preferred embodiments, the protein is heterologous to the host cell. In further preferred embodiments, the Gram-positive microorganism is a member of the genus Bacillus. In still further preferred embodiments, the Bacillus is selected from the group consisting of B. subtilis, B. licheniformis, B. lentus, B. brevis, B. stearothermophilus, B. alkalophilus, B. amyloliquefaciens, B. coagulans, B. circulans, B. lautus, and B. thuringiensis. In additional embodiments, the heterologous protein is selected from the group consisting of hormones, enzymes, growth factor, and cytokines. In some preferred embodiments, the heterologous protein is an enzyme. In some particularly preferred embodiments, the enzyme is selected from the group consisting of a proteases, cellulases, amylases, carbohydrases, lipases, isomerases, racemases, epimerases, tautomerases, mutases, transferases, kinases, and phosphatases.

### DESCRIPTION OF THE DRAWINGS

FIG. 1 provides the nucleic acid sequence (SEQ ID NO: 1) for secG and the amino acid sequence (SEQ ID NO: 2) of SecG.

FIG. 2 provides an amino acid alignment of the SecG sequence from E. coli (ecosecg.p1) (SEQ ID NO: 3), Haemophilus (haeinsecg.p1) (SEQ ID NO: 4), Mycoplasma (myclepsecg.p1) (SEQ ID NO: 5), B. subtilis (bsuyval.p1) (SEQ ID NO: 2), and the SecG consensus sequence (SEQ ID NO: 6) of these four organisms.

FIG. 3 provides the amino acid identity (consensus sequence: SEQ ID NO: 7) between B. subtilis SecG (SEQ ID NO: 2) and E. coli SecG (SEQ ID NO: 3).

FIG. 4 provides the amino acid identity between B. subtilis SecG (SEQ ID NO: 2) and Mycoplasma SecG (SEQ ID NO: 5).

FIG. 5 provides a hydrophilicity profile of B. subtilis SecG.

FIG. 6A provides results from a Coomassie stained SDS-PAGE of cell fractions of B. subtilis DB104 and DB104:ΔyvaL. Lower case “c” refers to cellular fraction; lower case “m” refers to medium. The position of a polypeptide band is indicated that is present in the wild-type cells, but absent in the deletion mutant.

FIG. 6B provides data from the proteinase K digestion of cell associated proteins. As indicated, the digestion of the polypeptide band at 30 kDa is absent in the DB104:ΔyvaL cells. The final lane shows a control with Triton X®-100, to demonstrate that proteinase K is present in excess amounts.

FIG. 7A provides results from a Coomassie stained SDS-PAGE of E. coli inner membrane vesicles expressing the B. subtilis SecYE and either E. coli SecG or B. subtilis SecG (YvaL) compared to wild type vesicles. The positions of B. subtilis SecY and SecE are indicated.

FIG. 7B provides an immunoblot developed with a pAb directed against a synthetic polypeptide of E. coli SecG.

FIG. 7C provides an immunoblot developed with a pAb directed against a synthetic polypeptide of B. sibtilis SecG.

FIG. 8 provides an in vitro translocation of ¹²⁵I-labelled prePhoB into E. coli inside out vesicles. Vesicles were stripped for SecA and purified B. subtilis SecA was added when indicated.

### DESCRIPTION OF THE INVENTION

The present invention generally relates to expression of proteins in Gram-positive microorganisms and specifically to the Gram-positive microorganism secretion factor, SecG. The present invention also provides expression vectors, methods and systems for the production of proteins in Gram-positive microorganisms.

The capacity of the secretion machinery of a Gram-positive microorganism may become a limiting factor or bottleneck to protein secretion and the production of proteins in secreted form, in particular when the proteins are recombinantly introduced and overexpressed by the host cell. The present invention provides a means for alleviating that bottle neck.

The present invention is based, in part, upon the discovery of a B. sibtilis SecG secretion factor (also referred to herein as YVAL) identified in heretofore uncharacterized translated genomic DNA by its homology with a consensus sequence for SecG (based upon SecG sequences for E. coli, Haemophilus, and Mycoplasma) and the demonstration that B. subtilis SecG is a functional homolog of E. coli SecG. The present invention is also based, in part, upon the determination that B. sibtilis SecG in combination with other B. subtilis secretion factors forms a functional preprotein translocase.

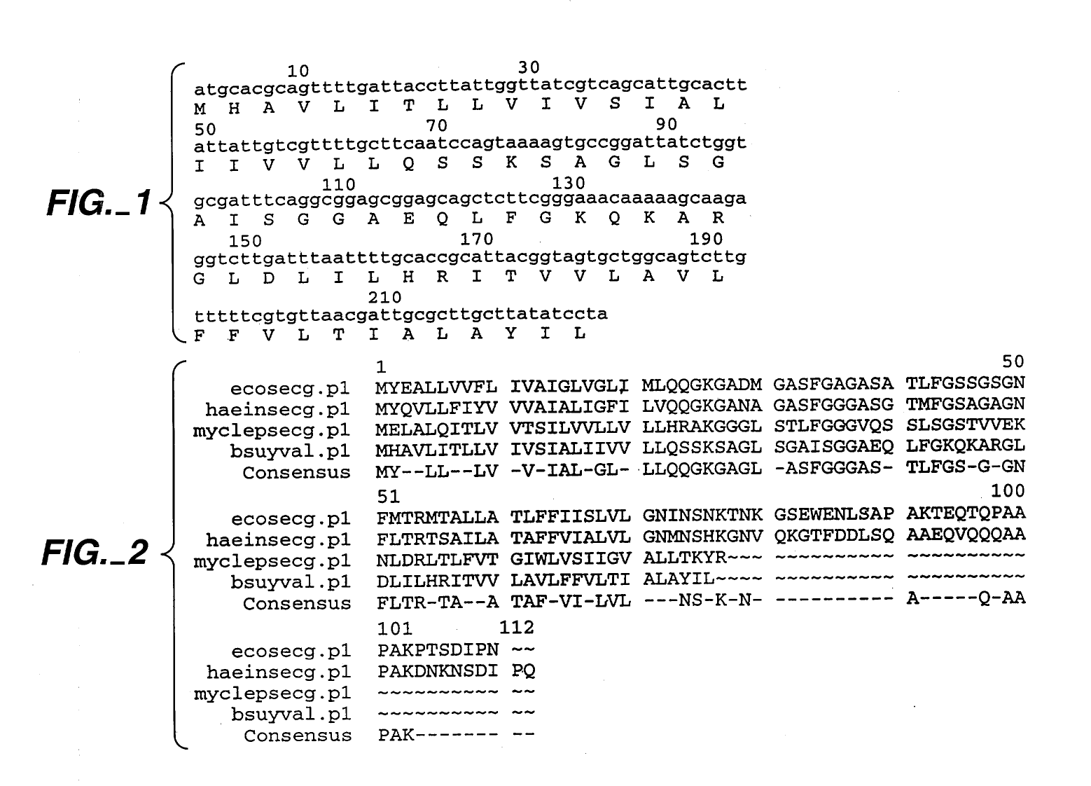

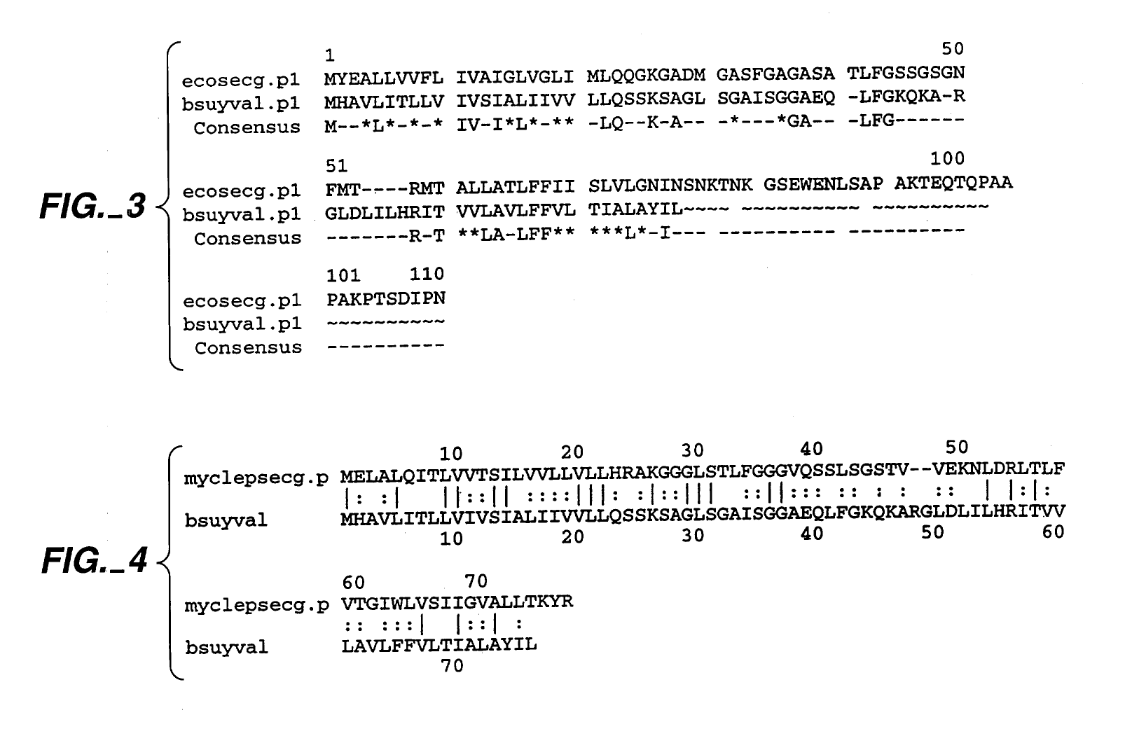

The present invention provides isolated nucleic acid and deduced amino acid sequences for B. sibtilis SecG. The amino acid sequence for B. sibtilis SecG (SEQ ID NO: 1) is shown in FIG. 1. The nucleic acid sequence encoding B. sibtilis SecG (SEQ ID NO: 2) is also shown in FIG. 1.

The present invention also provides improved methods for secreting proteins from Gram-positive microorganisms. Accordingly, the present invention provides improved methods for secreting a desired protein in a Gram-positive microorganism, comprising the steps of obtaining a Gram-positive microorganism host cell comprising nucleic acid encoding SecG wherein the nucleic acid is under the control of expression signals capable of expressing SecG in a Gram-positive microorganism, wherein the microorganism further comprises nucleic acid encoding the desired protein; culturing the microorganism under conditions suitable for expression of SecG; and then finally expressing and secreting the protein. In one embodiment of the present invention, the desired protein is homologous or naturally occurring in the Gram-positive microorganism. In another embodiment of the present invention, the desired protein is heterologous to the Gram-positive microorganism.

In one aspect of the present invention, a microorganism is genetically engineered to produce a desired protein, such as an enzyme, growth factor or hormone. In some preferred embodiments, the enzyme is selected from the group consisting of proteases, carbohydrases including amylases, cellulases, xylanases, and lipases; isomerases such as racemases, epimerases, tautomerases, or mutases, transferases, kinases, phosphatases, acylases, amidases, esterases, reductases, and oxidases. In further embodiments the expression of the secretion factor SecG is coordinated with the expression of other components of the secretion machinery. Preferably, other components of the secretion machinery (i.e., translocase, SecA, SecY, SecE and/or other secretion factors known to those of skill in the art) are modulated in expression at an optimal ratio to SecG. For example, in some embodiments, it is desirable to overexpress multiple secretion factors in addition to SecG for optimum enhancement of the secretion machinary. In one particular embodiment disclosed herein, B. sibtilis SecG is expressed along with B. sibtilis SecYE and SecA to form a functional preprotein translocase.

The present invention also provides method for identifying homologous Gram-positive microorganism SecG proteins. In some embodiments, the methods comprise hybridizing part or all of B. sibtilis SecG nucleic acid (e.g., as shown in FIG. 1; SEQ ID NO: 2) with nucleic acid derived from other Gram-positive microorganism(s) of interest. In one embodiment, the nucleic acid is of genomic origin, while in other embodiments, the nucleic acid is a cDNA. The present invention further encompasses novel Gram-positive microorganism secretion factors identified by this method.

The present invention also provides method and compositions for the mutagenesis of the chromosomal, native SecG promoter sequence. In some preferred embodiments, this mutagenesis results in increased or decreased transcription of the SecG gene. In still further embodiments, the Shine-Delgarno sequence (i.e., ribosome binding site) and/or RNA polymerase sigma factor alpha (σ_{A}) is mutated to increase or decrease the transcription/translation of the SecG transcript (See e.g., Henner, DNA 3:17-21 [1984]). Thus, in addition to methods utilizing expression vectors to modulate SecG expression, the present invention provides methods and compositions that involve modulation of the chromosomal, native SecG promoter.

### Detailed Description

Prior to providing a description of the invention, Applicants provide the following definitions.

Definitions

As used herein, the genus Bacillus includes all species and subspecies known to those of skill in the art, including but not limited to B. subtilis, B. licheniformis, B. lentus, B. brevis, B. stearothermophilus, B. alkalophilus, B. amyloliquefaciens, B. coagulans, B. circulans, B. lautus, and B. thuringiensis.

The present invention encompasses novel SecG secretion factors from Gram-positive microorganisms In a preferred embodiment, the Gram-positive organism is a member of the genus Bacillus. In another preferred embodiment, the Gram-positive organism is B. subtilis. As used herein, the phrase, “B. sibtilis SecG secretion factor” refers to the deduced amino acid sequence (SEQ ID NO: 1), as shown in FIG. 1. The present invention encompasses variants of the amino acid sequence disclosed in FIG. 1 that are able to modulate secretion alone or in combination with other secretions factors.

As used herein, “nucleic acid” refers to a nucleotide or polynucleotide sequence, and fragments or portions thereof, and to DNA or RNA of genomic or synthetic origin which may be double-stranded or single-stranded, whether representing the sense or antisense strand. As used herein “amino acid” refers to peptide or protein sequences or portions thereof.

As used herein, lower case “secG” is used to designate a nucleic acid sequence, whereas capitalized “SecG” is used to designate an amino acid sequence.

A “B. sibtilis polynucleotide homolog” or “polynucleotide homolog” as used herein refers to a novel polynucleotide that has at. least 80%, at least 90%, or in preferred embodiments, at least 95% identity to the secG polynucleotide (SEQ ID NO: 2) in FIG. 1 or a sequence which is capable of hybridizing to the polynucleotide (SEQ ID NO: 2) of FIG. 1 under conditions of high stringency and which encodes an amino acid sequence that is able to modulate secretion of the Gram-positive microorganism from which it is derived.

The term “gene of interest” as used herein refers to the gene inserted into the polylinker of an expression vector whose expression in the cell is desired for the purpose of performing further studies on the transfected cell. The gene of interest may encode any protein whose expression is desired in a transfected cell at high levels. The gene of interest is not limited to the examples provided herein; the gene of interest may include cell surface proteins, secreted proteins, ion channels, cytoplasmic proteins, nuclear proteins (e.g., regulatory proteins), mitochondrial proteins, etc.

As used herein, the term “modulate” refers to the increase or decrease in secretion or expression of a gene. In particularly preferred embodiments, the term refers to alteration(s) in the expression of secretion factor(s) to alter the secretion patterns of proteins.

The terms “isolated” and “purified” as used herein refer to a component (e.g., nucleic acid or amino acid) that is removed from at least one component with which it is naturally associated.

As used herein, the term “heterologous protein” refers to a protein or polypeptide that does not naturally occur in a Gram-positive host cell. Examples of heterologous proteins include enzymes such as hydrolases including proteases, cellulases, amylases, other carbohydrases, lipases, isomerases, racemases, epimerases, tautomerases, mutases, transferases, kinases, and phosphatases. In some embodiments, the heterologous gene encodes therapeutically significant proteins or peptides, such as growth factors, cytokines, ligands, receptors and inhibitors, as well as vaccines and antibodies. In some embodiments, the gene encodes commercially important industrial proteins or peptides, such as proteases, carbohydrases such as amylases and glucoamylases, cellulases, oxidases, and lipases. In some embodiments, the gene of interest is a naturally occurring gene, while in other embodiments, it is a mutated gene, and in still further embodiments, it is a synthetic gene.

The term “homologous protein” refers to a protein or polypeptide native or naturally occurring in a Gram-positive host cell. The invention includes host cells producing the homologous protein via recombinant DNA technology. The present invention encompasses a Gram-positive host cell having a deletion or interruption of the nucleic acid encoding the naturally occurring homologous protein, such as a protease, and having nucleic acid encoding the homologous protein, or a variant thereof re-introduced in a recombinant form. In another embodiment, the host cell produces the homologous protein.

The terms “recombinant protein” and “recombinant polypeptide,” as used herein refers to a protein molecule which is expressed from a recombinant DNA molecule.

The term “native protein” is used herein to indicate that a protein does not contain amino acid residues encoded by vector sequences (i.e., the native protein contains only those amino acids found in the protein as it occurs in nature). A native protein may be produced by recombinant means or may be isolated from a naturally occurring source.

As used herein the term “portion” when in reference to a protein (as in “a portion of a given protein”) refers to fragments of that protein. The fragments may range in size from four amino acid residues to the entire amino acid sequence minus one amino acid.

As used herein, the term “fusion protein” refers to a chimeric protein containing the protein of interest joined to an exogenous protein fragment. The fusion partner may enhance solubility of the protein as expressed in a host cell, may provide an affinity tag to allow purification of the recombinant fusion protein from the host cell or culture supernatant, or both. If desired, the fusion protein may be removed from the protein of interest by a variety of enzymatic or chemical means known to the art.

The term “modulate,” as used herein, refers to a change or an alteration in the biological activity of an enzyme. It is intended that the term encompass an increase or a decrease in protein activity, a change in binding characteristics, or any other change in the biological, functional, or immunological properties of an enzyme.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein, the term “vector” is used in reference to nucleic acid molecules that transfer DNA segment(s) from one cell to another. The term “vehicle” is sometimes used interchangeably with “vector.”

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes include a promoter, optionally an operator sequence, a ribosome binding site and possibly other sequences.

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Qβ replicase, MDV-1 RNA is the specific template for the replicase (Kacian et al., Proc. Natl. Acad. Sci. USA 69:3038 [1972]). Other nucleic acid will not be replicated by this amplification enzyme. Similarly, in the case of T7 RNA polymerase, this amplification enzyme has a stringent specificity for its own promoters (Chamberlin et al., Nature 228:227 [1970]). In the case of T4 DNA ligase, the enzyme will not ligate the two oligonucleotides or polynucleotides, where there is a mismatch between the oligonucleotide or polynucleotide substrate and the template at the ligation junction (Wu and Wallace, Genomics 4:560 [1989]). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences (Erlich (ed.), PCR Technology, Stockton Press [1989]).

As used herein, the term “amplifiable nucleic acid” is used in reference to nucleic acids that may be amplified by any amplification method. It is contemplated that “amplifiable nucleic acid” will usually comprise “sample template.”

As used herein, the term “sample template” refers to nucleic acid originating from a sample which is analyzed for the presence of “target” (defined below). In contrast, “background template” is used in reference to nucleic acid other than sample template which may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

In some embodiments of the present invention, a nucleic acid sequence of at least about 10 nucleotides and as many as about 60 nucleotides from the SecG nucleotide sequence is of FIG. 1, preferably about 12 to 30 nucleotides, and more preferably about 20-25 nucleotides find use as a probe or PCR primer.

As used herein, the term “target,” when used in reference to the polymerase chain reaction, refers to the region of nucleic acid bounded by the primers used for polymerase chain reaction. Thus, the “target” is sought to be sorted out from other nucleic acid sequences. A “segment” is defined as a region of nucleic acid within the target sequence.

As used herein, the term “polymerase chain reaction” (“PCR”) refers to the methods of U.S. Pat. Nos. 4,683,195 4,683,202, and 4,965,188, hereby incorporated by reference, which describe methods for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”.

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “amplification reagents” refers to those reagents (deoxyribonucleotide triphosphates, buffer, etc.), needed for amplification except for primers, nucleic acid template and the amplification enzyme. Typically, amplification reagents along with other reaction components are placed and contained in a reaction vessel (test tube, microwell, etc.).

As used herein, the terms “restriction endonucleases” and “restriction enzymes” refer to bacterial enzymes, each of which cut double-stranded DNA at or near a specific nucleotide sequence.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids.

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, “Quantitative Filter Hybridization,” in Nucleic Acid Hybridization[1985]). Other references include more sophisticated computations, which take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. With “high stringency” conditions, nucleic acid base pairing will occur only between nucleic acid fragments that have a high frequency of complementary base sequences. Thus, conditions of “weak” or “low” stringency are often required with nucleic acids that are derived from organisms that are genetically diverse, as the frequency of complementary sequences is usually less.

“Maximum stringency” typically occurs at about Tm−5° C. (5° C. below the Tm of the probe); “high stringency” at about 5° C. to 10° C. below Tm; “intermediate stringency” at about 10° C. to 20° C. below Tm; and “low stringency” at about 20° C. to 25° C. below Tm. As will be understood by those of skill in the art, a maximum stringency hybridization can be used to identify or detect identical polynucleotide sequences while an intermediate or low stringency hybridization can be used to identify or detect polynucleotide sequence homologs.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods which depend upon binding between nucleic acids.

The term “homology” refers to a degree of complementarity. There may be partial homology or complete homology (i.e., identity). A partially complementary sequence is one that at least partially inhibits a completely complementary sequence from hybridizing to a target nucleic acid is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target which lacks even a partial degree of complementarity (e.g., less than about 30% identity); in the absence of non-specific binding the probe will not hybridize to the second non-complementary target.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions which promote hybridization under conditions of high stringency (e.g., increasing the temperature of the hybridization and/or wash steps, the use of formamide in the hybridization solution, etc.).

When used in reference to a double-stranded nucleic acid sequence such as a cDNA or genomic clone, the term “substantially homologous” refers to any probe which can hybridize to either or both strands of the double-stranded nucleic acid sequence under conditions of low stringency as described above.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

As used herein a “deletion” is defined as a change in either nucleotide or amino acid sequence in which one or more nucleotides or amino acid residues, respectively, are absent. Altered Gram positive secG polynucleotide sequences which find use in the present invention include deletions, insertions or substitutions of different nucleotide residues resulting in a polynucleotide that encodes the same or a functionally equivalent secG homolog, respectively.

As used herein an “insertion” or “addition” is that change in a nucleotide or amino acid sequence which has resulted in the addition of one or more nucleotides or amino acid residues, respectively, as compared to the naturally occurring Gram positive secG. As used herein “substitution” results from the replacement of one or more nucleotides or amino acids by different nucleotides or amino acids, respectively.

### Detailed Description of the Preferred Embodiments

The present invention provides novel Gram-positive microorganism secretion factors and methods that can be used in Gram-positive microorganisms to ameliorate the bottleneck to protein secretion and the production of proteins in secreted form, in particular when the proteins are recombinantly introduced and overexpressed by the host cell. In particularly preferred embodiments, the present invention provides the secretion factor SecG derived from B. subtilis.

I. SecG Nucleic Acid and Amino Acid Sequences

A. SecG Nucleic Acid Sequences

The SecG polynucleotide having the sequence (SEQ ID NO: 2) as shown in FIG. 1 encodes the B. sibtilis secretion factor SecG. A FASTA search of B. sibtilis translated genomic sequences with the E. coli SecG sequence alone did not identify the B. sibtilis SecG. The B. sibtilis SecG was identified via a FASTA search of Bacillus subtilis translated genomic sequences using a consensus sequence of 30 amino acids of SecG derived from E. coli (SEQ ID NO: 3) Haemophilus (SEQ ID NO: 4) and Mycoplasma (SEQ ID NO: 5) species as shown in FIG. 2. The consensus sequence used was “LVGLILLQQG KGAXXGASFG GGASXTLFGS” (SEQ ID NO: 6), given in the amino terminus to carboxy terminus direction with the FASTA search (Release 1.0, released on Jun. 11, 1997) parameters being Scoring matrix: GenRunData: blosum50.cmp; variable pamfactor used; Gap creation penalty: 12; and Gap extension penalty: 2.

As indicated above, the present invention provides Gram-positive secG polynucleotides which may be used alone or together with other secretion factors, such as SecY, SecE and SecA, in a Gram-positive host cell for the purpose of increasing the secretion of desired heterologous or homologous proteins or polypeptides.

The present invention encompasses secG polynucleotide homologs encoding novel Gram-positive microorganism SecG whether encoded by one or multiple polynucleotides which have at least 80%, at least 90%, or at least 95% identity to B. sibtilis SecG, as long as the homolog encodes a protein that is able to function by modulating secretion in a Gram-positive microorganism. As will be understood by the skilled artisan, due to the degeneracy of the genetic code, a variety of polynucleotides (i.e., SecG polynucleotide variants), can encode the B. sibtilis secretion factors SecG. The present invention encompasses all such polynucleotides.

Gram-positive polynucleotide homologs of B. sibtilis SecG may be obtained by standard procedures known in the art from, for example, cloned DNA (e.g., a DNA “library”), genomic DNA libraries, by chemical synthesis once identified, by cDNA cloning, or by the cloning of genomic DNA, or fragments thereof, purified from a desired cell using methods known in the art (See, for example, Sambrook et al., Molecular Cloning, A Laboratory Manual, 2d Ed., Cold Spring Harbor Laboratory Press, Cold Spring Harbor, N.Y. [1989]; and Glover (ed.), DNA Cloning: A Practical Approach, MRL Press, Ltd., Oxford, U.K. Vol. I, II. [1985]). A preferred source of DNA is from genomic DNA. In some embodiments, nucleic acid sequences derived from genomic DNA contain regulatory regions in addition to coding regions. Whatever the source, it is contemplated that the isolated secG gene is molecularly cloned into a suitable vector for propagation of the gene.

In the molecular cloning of the gene from genomic DNA, DNA fragments are generated, some of which will encode the desired gene. The DNA may be cleaved at specific sites using various restriction enzymes. Alternatively, one may use DNAse in the presence of manganese to fragment the DNA, or the DNA can be physically sheared, as for example, by sonication. The linear DNA fragments can then be separated according to size by standard techniques, including but not limited to, agarose and polyacrylamide gel electrophoresis and column chromatography.

Once the DNA fragments are generated, identification of the specific DNA fragment containing the SecG may be accomplished in a number of ways. For example, a B. subtilis SecG gene of the present invention or its specific RNA, or a fragment thereof, such as a probe or primer, may be isolated and labeled and then used in hybridization assays to detect a Gram-positive SecG gene (See, Benton and Davis, Science 196:180 [1977]; and Grunstein and Hogness, Proc. Natl. Acad. Sci. USA 72:3961 [1975]). Those DNA fragments sharing substantial sequence similarity to the probe will hybridize under stringent conditions.

Accordingly, the present invention provides a method for the detection of Gram-positive SecG polynucleotide homologs which comprises hybridizing part or all of a nucleic acid sequence of B. sibtilis SecG with Gram-positive microorganism nucleic acid of either genomic or cDNA origin.

Also included within the scope of the present invention are Gram-positive microorganism polynucleotide sequences that are capable of hybridizing to the nucleotide sequence of B. sibtilis SecG under conditions of intermediate to maximal stringency. Hybridization conditions are based on the melting temperature (Tm) of the nucleic acid binding complex, as taught in Berger and Kimmel (“Guide to Molecular Cloning Techniques,” in Methods in Enzymology, vol. 152, Academic Press, San Diego Calif. [1987]) incorporated herein by reference, and confer a defined stringency.

Also included within the scope of the present invention are novel Gram-positive microorganism secG polynucleotide sequences that are capable of hybridizing to part or all of the secG nucleotide sequence of FIG. 1 under conditions of intermediate to maximal stringency.

B. Amino Acid Sequences

The B. sibtilis secG polynucleotide as shown in FIG. 1 encodes B. sibtilis SecG. The present invention encompasses novel Gram positive microorganism amino acid variants of the amino acid sequence shown in FIG. 1 that are at least 80% identical, at least 90% identical, or at least 95% identical to the sequence shown in FIG. 1, as long as the amino acid sequence variant is able to function by modulating secretion of proteins in Gram-positive microorganisms alone or in combination with other secretion factors.

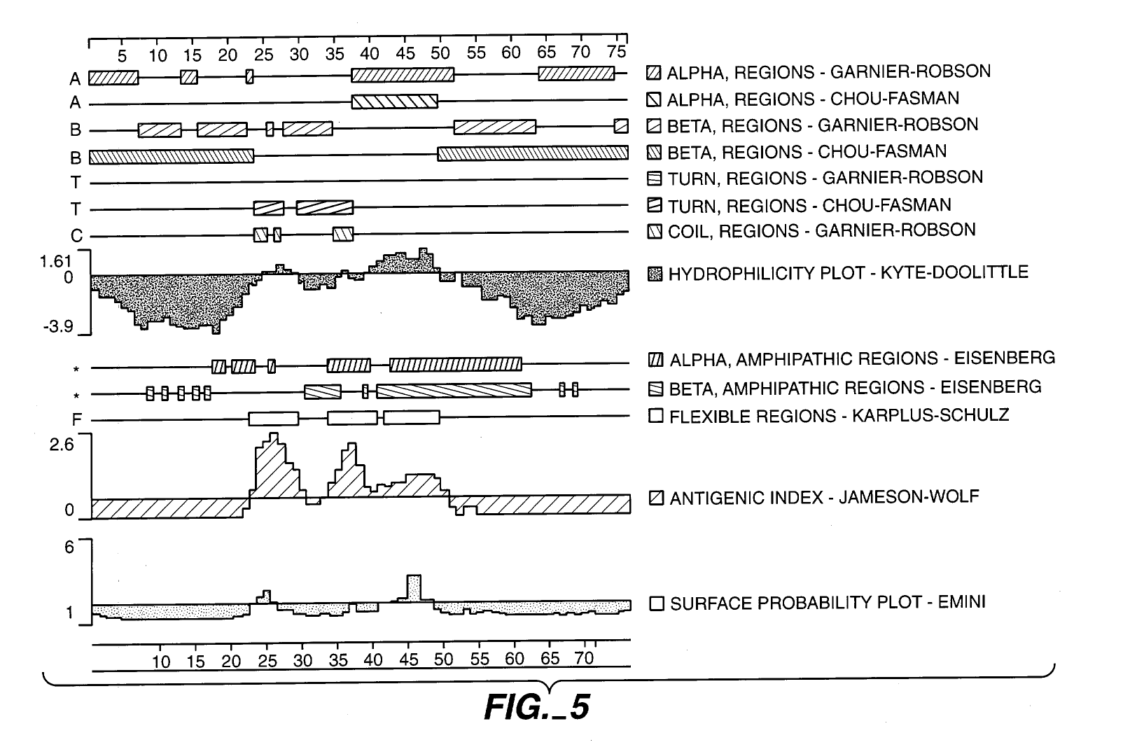

The secretion factor SecG as shown in FIG. 1 was subjected to a FASTA (Lipmann Pearson routine) amino acid search against a consensus amino acid sequence for SecG. The amino acid alignment is shown in FIG. 2. The hydrophilicity profile for B. sibtilis SecG as shown in FIG. 5 shows two potential membrane spanning regions.

II. Expression Systems

The present invention provides expression systems for the enhanced production and secretion of desired heterologous or homologous proteins in Gram-positive microorganisms.

A. Coding Sequences

In the present invention, the vector comprises at least one copy of nucleic acid encoding a Gram-positive microorganism SecG secretion factor and preferably comprises multiple copies. In a preferred embodiment, the Gram-positive microorganism is Bacillus. In another preferred embodiment, the Gram-positive microorganism is Bacillus subtilis. In a preferred embodiment, polynucleotides which encode B. sibtilis SecG, or fragments thereof, or fusion proteins or polynucleotide homolog sequences that encode amino acid variants of SecG, may be used to generate recombinant DNA molecules that direct the expression of SecG, or amino acid variants thereof, respectively, in Gram-positive host cells. In a preferred embodiment, the host cell belongs to the genus Bacillus. In another preferred embodiment, the host cell is B. subtilis.

As understood by those of skill in the art, in some embodiments, it is advantageous to produce polynucleotide sequences possessing non-naturally occurring codons. Codons preferred by a particular Gram-positive host cell (Murray et al., Nucl. Acids Res., 17:477-508 [1989]) can be selected, for example, to increase the rate of expression or to produce recombinant RNA transcripts having desirable properties, such as a longer half-life, than transcripts produced from naturally occurring sequence.

The encoded protein may also show deletions, insertions or substitutions of amino acid residues, which produce a silent change and result in a functionally equivalent Gram-positive secG variant. Deliberate amino acid substitutions may be made on the basis of similarity in polarity, charge, solubility, hydrophobicity, hydrophilicity, and/or the amphipathic nature of the residues as long as the variant retains the ability to modulate secretion. For example, negatively charged amino acids include aspartic acid and glutamic acid; positively charged amino acids include lysine and arginine; and amino acids with uncharged polar head groups having similar hydrophilicity values include leucine, isoleucine, valine; glycine, alanine; asparagine, glutamine; serine, threonine, phenylalanine, and tyrosine.

The secG polynucleotides of the present invention may be engineered in order to modify the cloning, processing and/or expression of the gene product. For example, mutations may be introduced using techniques, which are well known in the art (e.g., site-directed mutagenesis) to insert new restriction sites, to alter glycosylation patterns or to change codon preference, for example.

In one embodiment of the present invention, a secG polynucleotide is ligated to a heterologous sequence to encode a fusion protein. A fusion protein may also be engineered to contain a cleavage site located between the SecG nucleotide sequence and the heterologous protein sequence, so that the SecG protein may be cleaved and purified away from the heterologous moiety.

B. Vector Sequences

Expression vectors used in expressing the secretion factors of the present invention in Gram-positive microorganisms comprise at least one promoter associated with a Gram-positive SecG, which promoter is functional in the host cell. In one embodiment of the present invention, the promoter is the wild-type promoter for the selected secretion factor and in another embodiment of the present invention, the promoter is heterologous to the secretion factor, but still functional in the host cell.

Additional promoters associated with heterologous nucleic acid encoding desired proteins or polypeptides may be introduced via recombinant DNA techniques. In one embodiment of the present invention, the host cell is capable of overexpressing a heterologous protein or polypeptide and nucleic acid encoding one or more secretion factor(s) is(are) recombinantly introduced. In one preferred embodiment of the present invention, nucleic acid encoding SecG is stably integrated into the microorganism genome. In another embodiment, the host cell is engineered to overexpress a secretion factor of the present invention and nucleic acid encoding the heterologous protein or polypeptide is introduced via recombinant DNA techniques. Example III demonstrates that B. sibtilis SecG can be overexpressed in a host cell. The present invention encompasses Gram-positive host cells that are capable of overexpressing other secretion factors known to those of skill in the art, including but not limited to, SecA, SecY, SecE or other secretion factors known to those of skill in the art or identified in the future. In one embodiment disclosed herein in Example II, it is demonstrated that B. sibtilis SecG along with B. sibtilis secretion factors SecY, E, and A, is able to participate in forming a functional preprotein translocase.

In a preferred embodiment, the expression vector contains a multiple cloning site cassette which preferably comprises at least one restriction endonuclease site unique to the vector, to facilitate ease of nucleic acid manipulation. In a preferred embodiment, the vector also comprises one or more selectable markers. As used herein, the term selectable marker refers to a gene capable of expression in the Gram-positive host, which allows for ease of selection of those hosts containing the vector. Examples of such selectable markers include but are not limited to antibiotics, such as, erythromycin, actinomycin, chloramphenicol and tetracycline.

C. Transformation

In one embodiment of the present invention, nucleic acid encoding one or more Gram-positive secretion factor(s) of the present invention is introduced into a Gram-positive host cell via an expression vector capable of replicating within the host cell. Suitable replicating plasmids for Bacillus are known in the art (See e.g., Harwood and Cutting [eds.], Molecular Biological Methods for Bacillus, John Wiley & Sons [1990]; in particular, see chapter 3 [on plasmids], examples of suitable replicating plasmids for B. sibtilis are listed on page 92).

In other embodiments, nucleic acid encoding a Gram-positive micro-organism SecG is stably integrated into the microorganism genome. Preferred Gram-positive host cells included those within the genus Bacillus. Another preferred Gram-positive host cell is B. subtilis. As known in the art, several strategies have been described in the literature for the direct cloning of DNA in Bacillus. For example, plasmid marker rescue transformation involves the uptake of a donor plasmid by competent cells carrying a partially homologous resident plasmid (Contente et al., Plasmid 2:555-571 [1979]; Haima et al., Mol. Gen. Genet., 223:185-191 [1990]; Weinrauch et al., J. Bacteriol., 154(3):1077-1087 [1983]; and Weinrauch et al., J. Bacteriol., 169(3):1205-1211 [1987]). The incoming donor plasmid recombines with the homologous region of the resident “helper” plasmid in a process that mimics chromosomal transformation. In addition, methods for transformation by protoplast transformation are known in the art (See e.g., in Chang and Cohen, Mol. Gen. Genet 168:111-115 [1979]; Vorobjeva et al., FEMS Microbiol. Lett., 7:261-263 [1980]; Smith et al., Appl. Environ. Microbiol., 51:634 [1986]; Fisher et al., Arch. Microbiol., 139:213-217 [1981]; McDonald, Gen. Microbiol. 130:203 [1984]; Bakhiet et al., Appl. Environ. Microbiol., 49:577 [1985]; Mann et al., Curr. Microbiol., 13:131-135 [1985]; and Holubova, Folia Microbiol. 30:97 [1985]).

III. Identification of Transformants

Although the presence/absence of marker gene expression suggests that the gene of interest is also present, in preferred embodiments of the present invention, its presence and expression are confirmed. For example, if the nucleic acid encoding SecG is inserted within a marker gene sequence, recombinant cells containing the insert can be identified by the absence of marker gene function. Alternatively, a marker gene can be placed in tandem with nucleic acid encoding the secretion factor under the control of a single promoter. Expression of the marker gene in response to induction or selection usually indicates expression of the secretion factor as well.

Alternatively, host cells which contain the coding sequence for a secretion factor and express the protein may be identified by a variety of procedures known to those of skill in the art. These procedures include, but are not limited to, DNA-DNA or DNA-RNA hybridization and protein bioassay or immunoassay techniques, which include membrane-based, solution-based, or chip-based technologies for the detection and/or quantification of the nucleic acid or protein.

The presence of the secG polynucleotide sequence can be detected by DNA-DNA or DNA-RNA hybridization or amplification using probes, portions or fragments derived from the B. sibtilis secG polynucleotide.

IV. Secretion Assays

In an embodiment disclosed herein in Example IV, it is demonstrated that a B. subtilis cell having a disruption in nucleic acid encoding SecG appears to be defective in the secretion of some extracellular proteins.

Means for determining the levels of secretion of a heterologous or homologous protein in a Gram-positive host cell and detecting secreted proteins include, using either polyclonal or monoclonal antibodies specific for the protein to be detected. Examples include enzyme-linked immunosorbent assay (ELISA), radioimmunoassay (RIA) and fluorescent activated cell sorting (FACS). These and other immunoassay systems are known in the art (See e.g., Hampton et al., Serological Methods, a Laboratory Manual, APS Press, St Paul Minn. [1990]; and Maddox et al., J. Exp. Med., 158:1211 [1983]).

A wide variety of labels and conjugation techniques are known to those skilled in the art and can be used in various nucleic and amino acid assays. In addition, means for producing labeled hybridization or PCR probes for detecting specific polynucleotide sequences include oligolabeling, nick translation, end-labeling or PCR amplification using a labeled nucleotide. Alternatively, the nucleotide sequence, or any portion of it, may be cloned into a vector for the production of an mRNA probe. Such vectors are known in the art, are commercially available, and may be used to synthesize RNA probes in vitro by addition of an appropriate RNA polymerase such as T7, T3 or SP6 and labeled nucleotides.

A number of companies such as Pharmacia Biotech (Piscataway, N.J.), Promega (Madison Wis.), and US Biochemical Corp (Cleveland Ohio) supply commercial kits and protocols for these procedures. Suitable reporter molecules or labels include those radionuclides, enzymes, fluorescent, chemiluminescent, or chromogenic agents as well as substrates, cofactors, inhibitors, magnetic particles and the like. Patents teaching the use of such labels include US Pat. Nos. 3,817,837; 3,850,752; 3,939,350; 3,996,345; 4,277,437; 4,275,149 and 4,366,241, all of which are hereby incorporated by reference. Also, recombinant immunoglobulins may be produced as shown in US Pat. No. 4,816,567, and incorporated herein by reference.

V. Purification of Proteins

Gram-positive host cells transformed with polynucleotide sequences encoding heterologous or homologous protein may be cultured under conditions suitable for the expression and recovery of the encoded protein from cell culture. The protein produced by a recombinant Gram-positive host cell comprising a secretion factor of the present invention will be secreted into the culture media. Other recombinant constructions may join the heterologous or homologous polynucleotide sequences to nucleotide sequence encoding a polypeptide domain, which will facilitate purification of soluble proteins (See e.g., Kroll et al., DNA Cell Biol., 12:441-53 [1993]).

Such purification facilitating domains include, but are not limited to, metal chelating peptides such as histidine-tryptophan modules that allow purification on immobilized metals (Porath, Prot. Express. Purif., 3:263-281 [1992]), protein A domains that allow purification on immobilized immunoglobulin, and the domain utilized in the FLAGS extension/affinity purification system (Immunex Corp, Seattle Wash.). The inclusion of a cleavable linker sequence such as Factor XA or enrterokinasc (Invitrogen, San Diego Calif.) between the purification domain and the heterologous protein can also be used to facilitate purification.

The manner and method of carrying out the present invention may be more fully understood by those of skill in the art by reference to the following Examples. These Examples are not intended in any manner to limit the scope of the present invention or of the claims directed thereto. All publications and patents are hereby incorporated by reference in their entirety.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: M (molar); mM (millimolar); μM (micromolar); nM (nanomolar); mol (moles); mmol (millimoles); μmol (micromoles); nmol (nanomoles); gm (grams); mg (milligrams); μg (micrograms); pg (picograms); L (liters); ml (milliliters); μl (microliters); cm (centimeters); mm (millimeters); μm (micrometers); nm (nanometers); ° C. (degrees Centigrade); cDNA (copy or complimentary DNA); DNA (deoxyribonucleic acid); ssDNA (single stranded DNA); dsDNA (double stranded DNA); dNTP (deoxyribonucleotide triphosphate); RNA (ribonucleic acid); PBS (phosphate buffered saline); g (gravity); OD (optical density); Dulbecco's phosphate buffered solution (DPBS); HEPES (N-[2-Hydroxyethyl]piperazine-N-[2-ethanesulfonic acid]); HBS (HEPES buffered saline); SDS (sodium dodecylsulfate); Tris-HCl (tris[Hydroxymethyl]aminomethane-hydrochloride); Klenow (DNA polymerase I large (Klenow) fragment); rpm (revolutions per minute); EGTA (ethylene glycol-bis(β-aminoethyl ether) N, N, N′, N′-tetraacetic acid); EDTA (ethylenediaminetetracetic acid); bla (β-lactamase or ampicillin-resistance gene); Endogen (Endogen, Woburn, Mass.); Amersham (Amersham, Chicago, Ill.); DuPont NEN (DuPont NEN, Boston, Mass.); (Bio-Synthesis (Bio-Synthesis, Lewisville, Tex.); ATCC (American Type Culture Collection, Rockville, Md.); Gibco/BRL (Gibco/BRL, Grand Island, N.Y.); Sigma (Sigma Chemical Co., St. Louis, Mo.); Pharmacia (Pharmacia Biotech, Pisacataway, N.J.); Neosystem (Neosystem, Strasbourg, France); Schleicher & Schuell (Schleicher & Schuell, Durham N.H.); (US Biochemical (US Biochemical Corp, Cleveland, Ohio); Qiagen (Qiagen, Valencia, Calif.); and Stratagene (Stratagene, La Jolla, Calif.).

### EXAMPLE I

### Materials and Methods Used in Experiments Described in Examples II-VI.

A. Bacterial Strains and Growth Media

Strains were grown in Luria-Bertani Broth or on Luria-Bertani agar. When necessary, the medium was supplemented with relevant antibiotics as indicated. Construction of vectors was done in E. coli DH5α (supE44, Δlac U169, (Φ80lacZΔM15), hsdR17, recA1, endAI, gyrA96, thi-1, re1A1). Chromosomal deletions and growth experiments were done in B. subtilis DB104 (nprE18, aprEΔ3) as known in the art (See e.g., Yang et al., J. Bacteriol., 160:15-21 [1984]).

B. Construction of Plasmids

The E. coli secG and B. sibtilis yvaL genes including suitable ribosome binding sites were amplified as BamHI-XbaI cassettes by PCR from chromosomal DNA from strains DH5α and DB104, respectively, and cloned into pBluescript SK+, the primer used are listed in Table 1. The sequences of both open reading frames were determined and compared against relevant databases. For expression in E. coli, the genes were cloned into pET324 (Van der Does et al., Mol. Microbiol., 22:619-629 [1996]) yielding pET304 (E. coli secG) and pET820 (B. sibtilis yvaL).

Vectors pPR111 (a pUB110 derivative (See, Diderichsen et al., Plasmid 30:312-315 [1993]) and pBEY13 (a gift from Dr. R. Breitlin) are shuttle vectors using a ColE1 origin for replication in E. coli and RepR for replication in Gram-positive organisms. These plasmids encode ampicillin resistance markers for E. coli and phleomycin resistance markers for B. subtilis. Vector pBEY13 expresses the B. sibtilis secY and secE genes from the constitutive staphylococcal sak promoter. Plasmids pET470 and pET471 were formed by replacing the secYE cassette by E. coli secG and B. sibtilis yvaL, respectively. Vector pAMP21 is a pGK13 (Kok et al., Appl. Environ. Microbiol., 48: 726-731 [1984]) based broad host range vector containing the Lactococcus-derived p32 promoter (See, van der Vossen et al., Appl. Environ. Microbiol., 10:2452-2457 [1987]) with synthetic ribosome binding site and Ncol site overlapping the start codon. The B. amyloliquefaciens α-amylase gene was isolated by PCR from plasmid pKTH10 (See, Palva, Gene 1:81-87 [1982]) as an Ncol-BamHI cassette, and ligated into Ncol-BamHI digested pAMP21. The resulting vector, named pET468, harbors the amyQ gene under control of the constitutive p32 promoter. Vectors pET472 and pET473 were generated by ligating the E. coli and B. sibtilis secG genes, respectively, containing BamHI-BssHII fragments from the pBluescript derivatives into BamHI-BssHII-MluI digested pET468. Resulting vectors express B. amyloliquefaciens α-amylase and secG or yvaL as a tandem operon from the single p32 promoter.

A vector for the disruption of yvaL was generated as follows. The regions immediately upstream and downstream of the yvaL were amplified from chromosomal DNA from strain DB 104 as BamHI-XbaI and KpnI-HincII cassettes respectively, and cloned into pBluescript SK+. Subsequently, a BglII-PvuII digested chloramphenicol resistance marker was placed between the BamHI and HincII sites, yielding pDELG2. This vector contains the chromosomal region as is present in DB 104 with the yvaL replaced by the chloramphenicol resistance marker.

Plasmid pET812 containing a synthetic operon of Bacillus subtilis secy, secE and E. coli secG, and plasmid pET822 containing secy, and secE and yvaL of B. sibtilis were constructed for expression in E. coli as known in the art (See, Van der Does et al., [1996], supra) using the primers listed in Table 1.

The alkaline phosphates phoB (phoAIII) of B. sibtilis was amplified from chromosomal DNA of DB 104 using PCR (for primers see Table 1) and N-terminally fused to a his-tag using the plasmid pET302 (van der Does et al., Biochem., 37: 201-210 [1998]) so creating pET461. An overview of the plasmids used in this study is provided in Table 2.

TABLE 1
PCR Amplification Primers.

| | |
|---|---|
| Primer | Sequence |

| | | |
|---|---|---|
| B. subtilis secY forward | CGCCCATGGTTAAAAACAATCTCCAACTTTATGCG | (SEQ ID No:9) |
| | NcoI | |
| B. subtilis secY reverse | CGCGTCGACTTAGTTTTTCATAAATCCACGGTA | (SEQ ID No:10) |
| | ClaI | |
| B. subtilis secE forward | GGGATCGATGGAGGTTTTAATTCATGCGTATTATGAAA | (SEQ ID No:11) |
| | ClaI | |
| B. subtilis secE reverse | CGCGGATCCTCATTATTCAACTATTAA | (SEQ ID No:12) |
| | BamHI | |
| B. subtilis YvaL forward | AAAGGATCCTAGTCTGGAGGTGTATGGGATGC | (SEQ ID No:13) |
| | BamHI | |
| B. subtilis yvaL reverse | AAATCTAGATTCTCGAGCCCTATAGGATATAAGCAAGC | (SEQ ID No:14) |
| | XbaI | |
| E. coli secG forward | CCCGGATCCGGAGGTTTTAATTCATGTATGAAGCTCTTT | (SEQ ID No:15) |
| | BamHI | |
| E. coli secG reverse | CCCTCTAGACTCGAGTTAGTTCGGGATATCGC | (SEQ ID No:16) |
| | XbaI | |
| B. subtilis phoB forward | GGGCCATGGGAAAAAAATTCCCAAAGAAA | (SEQ ID No:17) |
| | NcoI | |
| B. subtilis phoB reverse | GGGGGATCCTTACTTATCGTTAATCTTAAT | (SEQ ID No:18) |
| | BamHI | |

In this Table, recognition sites of restriction enzymes used are underlined. Ribosome-binding sites, and start and stop codons are indicated in bold.

TABLE 2
List of Plasmids

| Name | Replicon | Resistance | Relevant Expression |
|---|---|---|---|
| pDELG2 | ColE1 | Amp, Cam | - (deletion vector) |
| pPR111 | ColE1, repR | Amp, Phleo | — |
| pET302 | pBR | Amp | — |
| pET304 | pBR | Amp | E. coli SecG |
| pET324 | pBR | Amp | — |
| pET461 | pBR | Amp | B. subtilis PhoB |
| | | | (his-tagged) |
| pET470 | ColE1, repR | Amp, Phleo | E. coli SecG |
| pET471 | ColE1, repR | Amp, Phleo | B. subtilis YvaL |
| pET468 | repA | Ery | α-amylase |
| pET472 | repA | Ery | α-amylase, E. coli SecG |
| pET473 | repA | Ery | α-amylase, B. subtilis YvaL |
| pET812 | pBR | Amp | B. subtilis SecYE |
| pET820 | pBR | Amp | B. subtilis YvaL |
| pET822 | pBR | Amp | B. subtilis SecYE-YvaL |

C. Deletion of SecG From the Chromosome of B. subtilis

Vector pDELG2 was digested with PvuII to yield a 2.8 kb linear fragment containing the regions flanking the yvaL, which was replaced by a chloramphenicol resistance marker. B. subtilis DB104 was transformed with the fragment using natural competence, as known in the art (See, Young, Nature 213:773-775 [1967]), and chloramphenicol resistant colonies were selected. The correct position of the chromosomal replacement was confirmed by PCR. In the resulting strain, DB104ΔG, the yvaL has been replaced by the chloramphenicol resistance gene while leaving the flanking regions intact.

D. Growth Experiments

B. sibtilis DB 104 and DB 104AG were transformed with each of six plasmids constructed for testing (i.e. pPR111, pET470, pET471, pET468, pET472 and pET473). After transformation, plates were incubated at 30° C. overnight. Selective pressure using the appropriate antimicrobial(s) was applied from this point onwards. No chloramphenicol was used at this stage. A single colony was picked for each transformant and cultured overnight at 30° C. in liquid medium. Then, 5μl of the overnight culture were inoculated on plates and incubated at temperatures ranging from 15° C. to 30° C., until the colonies of the wild-type strain reached a diameter of several millimeters. Plates were inspected daily and the occurrence and size of the colonies were noted.

For expression in E. coli plasmids pET820 and pET304 were transformed to E. coli KN370 (ΔsecG::kan) as described before (Nishiyama et al., EMBO J., 13:3272-3277 [1994]) and assay for the formation of single colonies on agar-plates at either 20° C. or at 37° C., with or without induction using IPTG (1mM).

E. Analysis of Secreted Proteins

B. sibtilis DB 104 and DB 104ΔG transformed with plasmid pET468 were grown overnight at 30° C. in liquid medium. The cultures were cooled on ice and fractionated into a cellular fraction and culture medium by centrifugation. Alternatively, the overnight cultures were diluted 1:50 into fresh medium, grown to an OD₆₀₀ of 0.6 and incubated overnight at 15° C. The culture supernatant was precipitated with 10% w/v TCA, washed twice with cold acetone and analyzed by SDS-PAGE. Cellular pellets of the cultures were resuspended in sample buffer, sonicated and analysed by SDS-PAGE. For further analysis of the cellular fractions, accessibility for proteinase K was tested. Transformed DB104 and DB104ΔG were grown overnight at 30° C. and harvested by centrifugation. The cellular pellet was washed once with TN (50 mM TRIS-C1, pH 7.5, 100 mM NaCl) buffer, and resuspended in the same buffer containing 0.5 mg/ml lysozyme. After incubation for 15 min. on ice, proteinase K was added to a final concentration ranging from 0 to 2 mg/ml and the suspension was incubated for an additional 15 min. Finally, the suspension was precipitated with TCA, washed with acetone and analyzed by SDS-PAGE.

F. Expression of pET812 and pET822 and Preparation of Inside Out Vesicles

E. coli SF100 was used for the overexpression of B. sibtilis SecY. SecE, and either SccG of E. coli (pET812) or YvaL of B. sibtilis (pET822). Expression of the proteins and isolation of inside out vesicles was performed as known in the art (See, Van der Does et al., [1996], supra).

G. E. coli SecA Stripping of the Vesicles and In Vitro Translocation

To remove the E. coli SecA from the inside out vesicles, 100 μl of vesicles (10 mg/ml) were incubated with 50 μl of polyclonal antibody directed against E. coli SecA (See, Schiebel et al., Mol. Microbiol., 22: 619-629 [1991]). In vitro translocation of ¹²⁵I-labeled his-prcPhoB (Van Wely et al., Eur.J. Biochem., 255:690-697 [1998]) into inner membrane vesicles was assayed as known in the art (See e.g., Van Der Does et al., [1996], supra) except that purified B. sibtilis SecA (Van der Wolk et al., Mol. Microbiol., 8:31-42 [1993]) was used instead of E. coli SecA (0.5 μg).

H. Production of B. sibtilis SecG Polyclonal Antibody

A peptide polyclonal antibody directed against the internal YvaL sequence Tyr-Ala-Glu-Gln-Leu-Phe-Gly-Lys-Gln-Lys-Ala-Arg-Gly-Leu-Asp (SEQ ID No: 19) coupled to KLH via the tyrosine residue was produced in rabbits according to standard procedures published by Neosystem.

### EXAMPLE II

### B. sibtilis SecG is a Functional Homolog of E. coli SecG

This Example describes experiments to determine whether B. sibtilis SecG is a functional homolog of E. coli SecG. The membrane vesicle derived from cells expressing pET812 and pET822 were stripped of their indigenous E. coli SecA using a polyclonal antibody directed against SecA and subjected to an in vitro translocation assay using ¹²⁵I-labeled his-prePhoB. In FIG. 8, the result of the translocation is shown. When no B. sibtilis SecA was added, both vesicles containing either SecYEG or SecYE and YVAL showed only little background translocation. However, when B. sibtilis SecA was added to vesicles containing SecYE and YVAL, an enormous increase in translocation efficiency of 1²⁵I-prePhoB was observed, while in the vesicles containing the SecYE and E. coli SecG no extra translocation is observed. From these data, it can be concluded that B. sibtilis SecYE, together with B. sibtilis Yval and SecA forms a functional preprotein translocase that mediates the translocation of Bacillus prePhoB protein in vitro.

### EXAMPLE III

### Over-Expression of Bacillus Proteins in E. coli

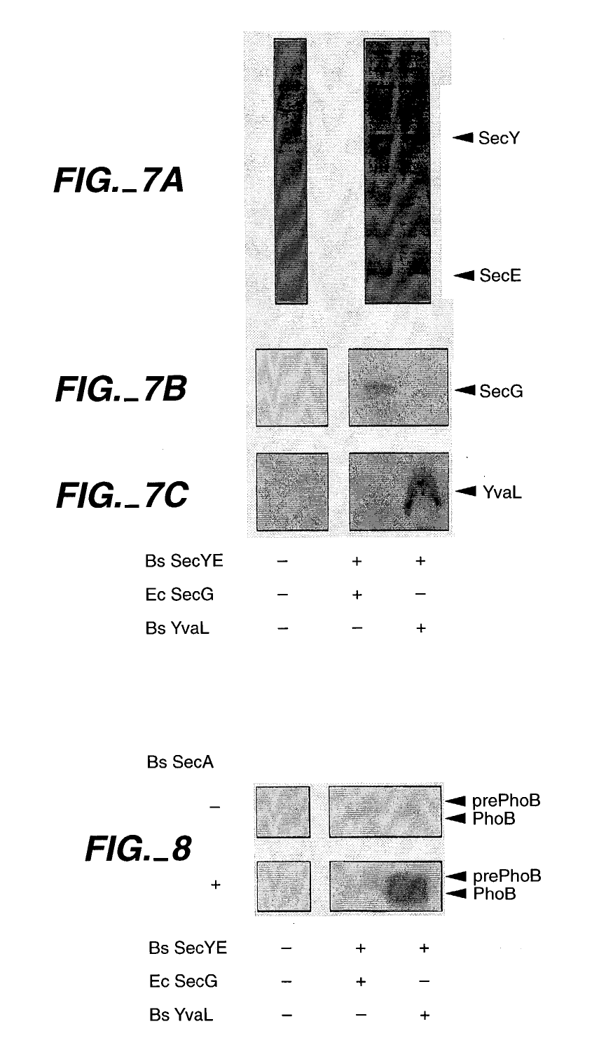

This Example illustrates that B. sibtilis SecY, SecE and SecG (YVAL) proteins can be overexpressed in E. coli. To establish whether the pET8 12 and pET 822 are expressed in E. coli SF100, inside out vesicles were analyzed on a 15% SDS-PAGE. Both the SecY and SecE of B. sibtilis were readily visible on a commassie stained gel (See, FIG. 7A). The B. subtilis SecG and increased amounts of E. coli SecG could be detected on an immunoblot using antibodies directed against these proteins, as indicated in FIGS. 7B-7C.

### EXAMPLE IV

### Secretion of Proteins

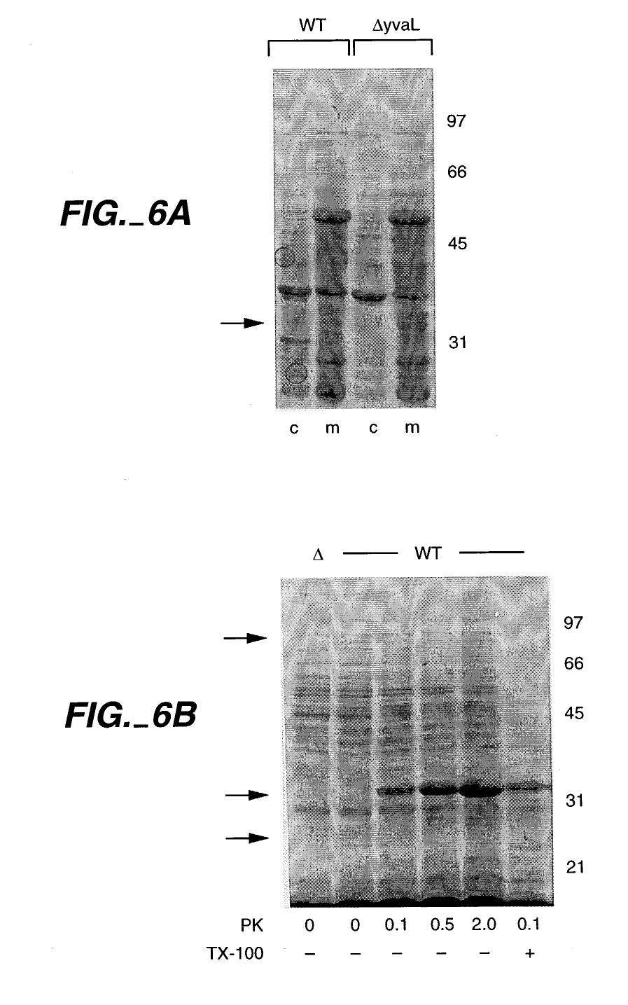

This Example illustrates the involvement of protein secretion machinery in the secretion of proteins for wild type cells and cells having a deletion in B. sibtilis SecG. In the culture supernatants of cells grown at different temperatures, no differences between wild type and mutant cells was observed (See, FIG. 6A). The cellular fraction, showed some differences in the banding pattern. The difference mainly concerns the absence of some bands in the mutant. The localization of these proteins was determined by breakdown of the cell wall by lysozyme and subsequent protease digestion of the accessible proteins (FIG. 6B). Some of the protein bands are digested already by low concentrations of proteinase K, whereas breakdown of most other proteins only occurs after disruption of the cell membrane by Triton X-100. These proteins appear to be secreted. Some of these secreted proteins are absent in the mutant strain. Therefore, the B. sibtilis SecG disruption mutant appears to be defective in the secretion of some extracellular proteins.

### EXAMPLE V

### Effect of SecG Deletion

This Example illustrates the effect of a SecG deletion on cell growth. Disruption of the E. coil secG gene has been shown to result in a cold-sensitive phenotype (See, Nishiyama et al., EMBO J., 13:3272-3277 [1994]), at non-permissive temperatures of 25° C. and below. Deletion of B. sibtilis secG from the chromosome did not result in any phenotype when cells were grown at 37° C. either on rich or minimal media. Incubations below 20° C. demonstrated a mild cold sensitivity, where the DB104ΔG strain showed progressively slower growth as compared to DB104. However, the mutant strain did not completely stop growing. Compared to the wild type, growth was retarded more severely when temperatures were lowered further. After shifting the cells again to higher temperatures, growth resumed at a faster rate.

Cells were transformed with plasmids expressing E. coli SecG or B. sibtilis SecG as well as a control plasmid. After preincubation at temperatures that do not affect growth of the mutant, cells were plated and incubated at several lower temperatures. Growth of the colonies was monitored over a period of several days. Wild type and mutant cells transformed with the control plasmid behaved like the non-transformed counterparts, showing retarded growth but not a complete stop at lower temperatures. Transformation of the mutant with pET471 expressing the secG gene product could relieve the retardation, showing that the phenotype of the mutant was not caused by any polar effects but by the deletion of secG itself. Surprisingly, when the mutant was transformed with pET470 expressing E. coli SecG, growth was stopped completely at temperatures of 20° C. or less. When the same plasmid was brought into the wild type cells, some interference with growth was observed at lower temperatures but not at 25° C. Thus, a disruption of the secG gene renders B. sibtilis mild cold-sensitive, but this is not an essential gene for B. subtilis. The results of these growth experiments are presented in Table 3, below.

TABLE 3
Results of Growth Experiments

| | | Growth at: | | |
|---|---|---|---|---|
| Strain: | Expression | 15° C. | 20° C. | 25° C. |
| DB104 :: 111 | — | ++ | ++ | ++ |
| DB104 :: 470 | E. coli SecG | ± | ± | ++ |
| DB104 :: 471 | B. sub YvaL | ++ | ++ | ++ |
| ΔyvaL :: 111 | — | ± | ± | ++ |
| ΔyvaL :: 470 | E. coli SecG | − | − | ++ |
| ΔyvaL :: 471 | B. sub YvaL | ++ | ++ | ++ |
| DB104 :: 468 | α-amylase | ++ | ++ | ++ |
| DB104 :: 472 | α-amylase, E. coli SecG | ± | ± | ++ |
| DB104 :: 473 | α-amylase, B. sub YvaL | ++ | ++ | ++ |
| ΔyvaL :: 468 | α-amylase | − | − | ++ |
| ΔyvaL :: 472 | α-amylase, E. coli SecG | ± | ± | ± |
| ΔyvaL :: 473 | α-amylase, B. sub YvaL | ± | ± | ± |

++, growth like reference strain;
±, growth, but slower than reference strain;
—, no growth.

### EXAMPLE VI

### Expression Effects

This Example describes the effects of expression of a secretory protein. B. subtilis cells mutant in secG and wild type cells were transformed with plasmid pET468 and derivatives. These plasmids express alpha-amylase, thereby invoking secretory stress. Derivatives of pET472 and pET473 express alpha amylase in combination with E. coli SecG or B. sibtilis SecG, respectively. Expression of alpha-amylase did not retard growth of the deletion mutant at 30° C., the temperature used for preculturing the cells. At this temperature, the halos that are formed by the alpha-amylase on starch-containing plates by transformants of wild type and deletion mutants were the same size. When pET468 transformants of the deletion mutant were shifted to lower temperatures, a clear and complete cold sensitivity was demonstrated. Already at 20° C., cells stopped growing completely. When the cells were transformed back to the permissive temperature of 30° C., after prolonged incubation at 20° C., growth was not resumed. Thus, the deletion mutant is capable of sustaining a basic level of secretion even at lower temperatures, but cannot handle overexpression of a secreted protein over a broad temperature range.

### EXAMPLE VII

### Identification of SecG Protein

This Example describes the detection of SecG in Gram-positive microorganisms.

DNA derived from a Gram-positive microorganism is prepared as known in the art. (according to the methods disclosed in Current Protocols in Molecular Biology, Chap. 2 or 3. The nucleic acid is subjected to hybridization and/or PCR amplification with a probe or primer derived from SecG. A preferred probe comprises the nucleic acid section containing conserved amino acid sequences

The nucleic acid probe is labeled by combining 50 pmol of the nucleic acid and 250 mCi of [gamma ³²p] adenosine triphosphate (Amersham) and T4 polynucleotide kinase (DuPont NEN). The labeled probe is purified with Sephadex G-25 super fine resin column (Pharmacia). A portion containing 10⁷ counts per minute of each is used in a typical membrane based hybridization analysis of nucleic acid sample of either genomic or cDNA origin.

The DNA sample which has been subjected to restriction endonuclease digestion is fractionated on a 0.7 percent agarose gel and transferred to nylon membranes (Nytran Plus, Schleicher & Schuell). Hybridization is carried out for 16 hours at 40° C. To remove nonspecific signals, blots are sequentially washed at room temperature under increasingly stringent conditions up to 0.1×saline sodium citrate and 0.5% sodium dodecyl sulfate. The blots are exposed to film for several hours, the film developed and hybridization patterns are compared visually to detect polynucleotide homologs of B. sibtilis SecG. The homologs are subjected to confirmatory nucleic acid sequencing. Methods for nucleic acid sequencing are well known in the art. Conventional enzymatic methods employ DNA polymerase Klenow fragment, SEQUENASE® (US Biochemical) or Taq polymerase to extend DNA chains from an oligonucleotide primer annealed to the DNA template of interest.

### EXAMPLE VIII

### Construction of B. sibtilis Host Cells Containing Mutant SecG Promoter

As indicated above, and described in greater detail herein, the level of the SecG protein produced after modifying the secG promoter may be modulated by changing either the chromosomal promoter or ribosome binding site to more or less closely match the RNA polymerase sigma factor A (σ_{A}) consensus sequence to affect transcription or the consensus Shine Delgarno sequence to affect translation.

The following sequence (SEQ ID NO: 20) provides the nucleic acid sequence of the SecG promoter, including 200 bp upstream and 200 bp downstream of the sequence, with the sequence elements targeted for nucleotide changes, the RNA polymerase sigma factor A (σ_{A}) promoter and Shine Delgarno ribosome binding site, underlined.

(SEQ ID NO:20)

```
            tcttcataaaaaagatgtttcctgctgtctatgctgata
            agcggcatcgcttttctcctttgaccttttcatatgaat
            agggtaaccaagataaaacgtcttatccggccttttggc
            gtctgatacagcgtgacatgccaacccttttcatgtaaa
            atagaagtaatgtagccagtgagtctggaggtgtatggg
    1 - atg cac gca gtt ttg att acc tta ttg gtt
  31 - atc gtc agc att gca ctt att att gtc gtt
  61 - ttg ctt caa tcc agt aaa agt gcc gga tta
  91 - tct ggt gcg att tca ggc gga gcg gag cag
121 - ctc ttc ggg aaa caa aaa gca aga ggt ctt
151 - gat tta att ttg cac cgc att acg gta gtg
181 - ctg gca gtc ttg ttt ttc gtg tta acg att
211 - gcg ctt gct tat atc cta
            tagggcaatgtttgtataaggtctgatgtgaagtcaggc
            ctttttcacgtttctggatgatattcaaaacgttttttt
            ctgattaaactgtggaaaactaaaatgatcgtgcagata
            gaaagggagacatgagcatgaaagttgtgacaccaaaac
            catttacatttaaaggcggagacaaagcggtgcttttgc
            tgcat
```

### Mutation of the Shine Delarno Site

As indicated herein, for mutation of the Shine Delgarno site, the sequence is altered to exactly match the consensus, changing the native sequence AGTCTGGAGGTGT (SEQ ID NO: 21) to AGAAAGGAGGTGA (SEQ ID NO: 22). The following description provides methods suitable for the mutation of the Shine Delgarno site.

### Construction of a PCR Fusion Sequence, Designated Herein as BC4

BC4 PCR fusion is constructed in three steps: 1) amplification of two separate fragments by PCR from B. sibtilis 168 chromosomal DNA; 2) assembly of two purified PCR fragments in PCR type process without primers; and 3) amplification of the assembled product by PCR with BCBS-1 and BCBS-8 end primers.

First, chromosomal B. sibtilis strain 168 DNA is used as a template for amplification of secG gene locus using two sets of primers. The first pair of primers consists of BCBS-1 located 3Kb 3′(downstream) of secG on the Bacillus chromosome and BCBS-2f (5′-ATAGAAGTAATGTAGCCAGTGAGAAAGGAGGTGAATGGGATGCACGCAGTTTTG-3′; SEQ ID NO: 23). The second pair of primers consists of BCBS-2r (5′-CAAAACTGCGTGCATCCCATTCACCTCCTTTCTCACTGGCTACATTACTTCTAT-3′; SEQ ID NO: 24), the reverse complement of BCBS-2r, and BCBS-3, located 3Kb 5′(upstream) of secG on the Bacillus chromosome. Both PCR products are overlapping in the promoter area of secG. BCBS-2f and BCBS-2r complementary primers are used for introduction of 4 mutations in the Shine Delgarno sequence, where AGTCTGGAGGTGT (SEQ ID NO: 21) was replaced with AGAAAGGAGGTGA (SEQ ID NO: 22) sequence. Standard PCR reactions using GeneAmp XL PCR kit containing rTth polymerase are used according to the manufacturer instructions for all PCRs. PCR reactions are performed in 100 μl volume.

DNA- 2-5 μl

3.3×XL Buffer II-30 μl

10 mM dNTP Blend-3 μl

25 mM Mg(OAc)2-4 μl

25 uM BCBS-1 primer (or BCBS-3)-2 μl

25 uM BCBS-2f primer (or BCBS-2r)-2 μl

2U/ul rTth polymerase-2 μl

Water- adjust to 100 μl

The PCR conditions are: 95° C.-30 sec, 54° C.-30 sec, 68° C.-3 min for 30 cycles. The obtained PCR fragments, 3 kb each, are purified with QIAGEN PCR purification kit according to the manufacturer instructions and used for PCR assembly.

In step 2, 5 μl aliquots of purified PCR fragments are mixed together and added into fresh PCR mix that didn't contain primers. The total volume of PCR mixture is 100 μl with components as described above. The PCR assembly conditions are: 95° C.-30 sec, 52° C.-30 sec, 68° C.-2 min for 10 cycles.

In step 3, after 10 cycles of PCR, the assembly mixture is supplemented with BCBS-1 and BCBS-3 primers and PCR amplification is run for 15 additional cycles. The PCR conditions this time are: 95° C.-30 sec, 52° C.-30 sec, 68° C.-5 min.

The desired 6 kb fusion product is then isolated and cloned into a standard integration vector such as pJM103 (See, Perego, in Sonenshein et al. (eds.), Bacillus subtilis and Other Gram-Positive Bacteria, chapter VI. 42, American Society for Microbiology, [1993]). SecG wild type strain of B. sibtilis is then transformed with the resulting recombinant plasmid, selecting, in the case of pJM103, for resistance to chloramphenicol, resulting in a strain carrying two copies of the 6 KB region, one with secG with a wild type Shine Delgarno, the second with the mutant sequence, separated by vector sequence, including the chloramphenicol resistance gene. After passage of the transformant in liquid broth culture in the absence of selection with chloramphenicol for multiple generations, chloramphenicol sensitive strains are recovered which have lost the duplicated region and vector sequences, approximately half of which will be the desired mutant. Wild type and mutant strains are distinguishable by PCR amplification of the region and DNA sequencing of the secG region using appropriate primers.

### Mutation of the RNA Polymerase σ_{A} Promoter Site

As indicated herein, for mutation of the σ_{A} promoter site, the sequence is altered to exactly match the consensus, changing the native sequence GTGACATGCCAACCCTTTTCATGTAAAAT (SEQ ID NO: 25) to TTGACATGCCAACCCTTTTCATGTATAAT (SEQ ID NO: 26), where the first six nucleotides in bold are the consensus-35 promoter sequence and the last six nucleotides in bold are the consensus-10 promoter sequence.

The methods described above for the mutation of the Shine Delgarno sequence find use in the mutation of the σ_{A} promoter site. However, primers BCBS-2f and BCBS-2r are replaced by the following primers:

| | |
|---|---|
| 4f: | |
| CTTTTGGCGTCTGATACAGCTTGACATGCCAACCCTTTTCATGTATAATA | (SEQ ID NO:27) |
| GAAGTAATGTAGCCAG | |
| 4r: | |
| CTGGCTACATTACTTCTATTATACATGAAAAGGGTTGGCATGTCAAGCTG | (SEQ ID NO:28) |
| TATCAGACGCCAAAAG | |

Various other examples and modifications of the foregoing description and examples will be apparent to a person skilled in the art after reading the disclosure without departing from the spirit and scope of the invention, and it is intended that all such examples or modifications be included within the scope of the appended claims. All publications and patents referenced herein are hereby incorporated in their entirety.

1. A Gram-positive microorganism encoding a mutated Shine Delgarno sequence such that the translation of the transcript comprising secretion factor G (SecG) is modulated.

2. The Gram-positive microorganism of claim 1, wherein said Gram-positive microorganism is a member of the genus Bacillus.

3. The Gram-positive microorganism of claim 2, wherein said Bacillus is selected from the group consisting of B. subtilis, B. licheniformis, B. lentus, B. brevis, B. stearothermophilus, B. alkalophilus, B. amyloliquefaciens, B. coagulans, B. circulans, B. lautus and B. thuringiensis.

4. The Gram-positive microorganism of claim 1, wherein said modulation comprises increasing the expression of said SecG.

5. The Gram-positive microorganism of claim 1, wherein said modulation comprises decreasing the expression of said SecG.

6. The Gram-positive microorganism of claim 1, wherein said microorganism is capable of expressing at least one heterologous protein.

7. The Gram-positive microorganism of claim 6, wherein said heterologous protein is selected from the group consisting of hormones, enzymes, growth factors, and cytokines.

8. The Gram-positive microorganism of claim 7, wherein said heterologous protein is an enzyme.

9. The Gram-positive microorganism of claim 8, wherein said enzyme is selected from the group consisting of a proteases, cellulases, amylases, carbohydrases, lipases, reductases, isomerases, epimerases, tautomerases, transferases, kinases, and phosphatases.

10. A Gram-positive microorganism encoding a mutated RNA polymerase sigma factor alpha (σ_{A}) sequence such that the expression of secretion factor G (SecG) is modulated.

11. The Gram-positive microorganism of claim 10, wherein said Gram-positive microorganism is a Bacillus.

12. The Gram-positive microorganism of claim 11, wherein the Bacillus is selected from the group consisting of B. subtilis, B. licheniformis, B. lentus, B. brevis, B. stearothermophilus, B. alkalophilus, B. amyloliquefaciens, B. coagulans, B. circulans, B. lautus and B. thuringiensis.

13. The Gram-positive microorganism of claim 10, wherein said modulation comprises increasing the expression of said SecG.

14. The Gram-positive microorganism of claim 10, wherein said modulation comprises decreasing the expression of said SecG.

15. The Gram-positive microorganism of claim 10, wherein said microorganism is capable of expressing at least one heterologous protein.

16. The Gram-positive microorganism of claim 15, wherein said heterologous protein is selected from the group consisting of hormones, enzymes, growth factors, and cytokines.

17. The Gram-positive microorganism of claim 16, wherein said heterologous protein is an enzyme.

18. The Gram-positive microorganism-of claim 17, wherein said enzyme is selected from the group consisting of a proteases, cellulases, amylases, carbohydrases, lipases, reductases, isomerases, epimerases, tautomerases, transferases, kinases, and phosphatases.

19. An improved method for secreting a protein in a Gram-positive microorganism comprising the steps of obtaining a Gram-positive microorganism host cell comprising nucleic acid encoding SecG wherein said nucleic acid is under the control of expression signals capable of expressing SecG in a Gram-positive microorganism and further comprising nucleic acid encoding said protein; and culturing said microorganism under conditions suitable for expression of SecG and expression and secretion of said protein.

20. The method of claim 19, wherein said Gram-positive microorganism further comprises nucleic acid encoding at least one additional secretion factor selected from the group consisting of SecY, SecE and SecA.

21. The method of claim 19, wherein said protein is homologous to said host cell.

22. The method of claim 19, wherein said protein is heterologous to said host cell.

23. The method of claim 19, wherein said Gram-positive microorganism is a member of the genus Bacillus.

24. The method of claim 23, wherein said Bacillus is selected from the group consisting of B. subtilis, B. licheniformis, B. lentus, B. brevis, B. stearothermophilus, B. alkalophilus, B. amyloliquefaciens, B. coagulans, B. circulans, B. lautus, and B. thuringiensis.

25. The method of claim 19, wherein said heterologous protein is selected from the group consisting of hormones, enzymes, growth factors, and cytokines.

26. The method of claim 25, wherein said heterologous protein is an enzyme.

27. The method of claim 26, wherein said enzyme is selected from the group consisting of a proteases, cellulases, amylases, carbohydrases, lipases, isomerases, racemases, epimerases, tautomerases, mutases, transferases, kinases, and phosphatases.
